(19) United States
(12) Patent Application Publication
(10) Pub. No.: US 2009/0216184 A1
(43) Pub. Date: Aug. 27, 2009

(54) DRUG STORAGE AND DELIVERY DEVICE

(75) Inventors: Bo Radmer, Hillerød (DK); Gunner Sie, Virum (DK); Morten Revsgaard, København (DK); Poul Larsen, Allerød (DK); Per Valentin Nielsen, København (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/281,533

(22) PCT Filed: Feb. 26, 2007
(86) PCT No.: PCT/EP07/51790

§ 371 Date: Feb. 16, 2009

(30) Foreign Application Priority Data
Mar. 7, 2006 (EP) 06004549.9
Aug. 18, 2006 (EP) 06119150.8

(51) Int. Cl. A61M 5/31; A61J 1/20

(52) U.S. Cl. 604/86; 604/416; 604/414

(57) ABSTRACT

A drug storage and delivery device (1) comprising a reservoir (2) containing a liquid, a vial (3) containing a dry drug, such as a lyophilized drug, means (6) for forcing the liquid from the reservoir (2) to the vial (3), via an established fluid connection, and locking means adapted to prevent the means (6) for establishing a fluid connection from re-establishing a previously disconnected fluid connection between the reservoir and the vial. Thereby the dry drug is reconstituted, and the forcing means (6) may subsequently be used for forcing reconstituted drug out of the device (1). The reservoir (2), the vial (3), the fluid connection and the forcing means (6) may form an at least substantially integral unit. This is an advantage because the number of steps needed to be performed by the user is reduced, the risk of contamination is reduced, the risk of incorrect reconstitution and dosage is reduced, and the device is easy to operate, e.g. using just one hand.

### FIELD OF THE INVENTION

The present invention relates to a drug storage and delivery device for reconstituting and delivering a dry drug, such as a lyophilized drug. More particularly, the present invention relates to such a drug storage and delivery device which is suitable for being operated using one hand.

### BACKGROUND OF THE INVENTION

For various reasons it is sometimes necessary or desirable to store a drug in dry form, such as in lyophilized form. This may, e.g., be in order to reduce degradation of the drug during storage. Before the drug is delivered to a person it must be reconstituted, i.e. the drug must be mixed with a liquid to form a liquid drug. The reconstituted drug can then be delivered to the person.

Various devices for reconstituting dry drugs are known. In most cases a normal syringe is used for applying a liquid to a vial containing the dry drug by means of a normal needle. The drug is thereby reconstituted, and afterwards the reconstituted drug is retrieved to the syringe. The reconstituted drug is then delivered to a person from the syringe by means of the needle. During this process there is a relatively high risk of contamination of the drug and/or of the needle. Furthermore, it is necessary for the person to use both hands in order to operate the syringe.

U.S. Pat. No. 6,689,108 discloses a system for reconstituting a lyophilized drug, and for delivering the reconstituted drug to a person. The system comprises a first port that receives a first container that contains a powdered lyophilized drug, and a second port that receives a second container that contains a fluid to be mixed with material in the first container, to form an injectable fluid. The system further comprises a channel that provides fluid communication between the first and second ports. During use of the system, a first container containing a lyophilized drug is positioned in the first port and a second container containing a fluid is positioned in the second port. Fluid communication is then established between the first and second ports in order to allow the fluid to enter the first container, thereby causing the lyophilized drug to become reconstituted. Care must be taken to ensure that the correct containers are used, and that they are positioned correctly.

### SUMMARY OF THE INVENTION

It is, thus, an object of the invention to provide a drug storage and delivery device for reconstituting dry drug, the device being operable using one hand.

It is a further object of the invention to provide a drug storage and delivery device for reconstituting dry drug, where the device can be delivered as an ‘all-in-one’ package.

It is an even further object of the invention to provide a drug storage and delivery device for reconstituting dry drug, wherein the risk of contamination of the reconstituted drug is reduced as compared to prior art devices.

It is an even further object of the invention to provide a drug storage and delivery device for reconstituting dry drug, in which the number of steps necessary to be performed by a user can be reduced as compared to prior art devices.

According to a first aspect of the invention the above and other objects are fulfilled by providing a drug storage and delivery device comprising:
a reservoir containing a liquid,
a vial containing a dry drug,
means for establishing a fluid connection between the reservoir and the vial,
first forcing means for forcing the liquid from the reservoir to the vial, via an established fluid connection, thereby causing the dry drug to become reconstituted, and
locking means adapted to prevent the means for establishing a fluid connection from re-establishing a previously disconnected fluid connection between the reservoir and the vial.

The reservoir may be any suitable kind of reservoir, such as a syringe, a flexible reservoir, e.g. a bag, etc. The liquid contained in the reservoir is preferably a solvent liquid which is suitable for reconstituting the dry drug contained in the vial.

As mentioned above, the dry drug may, e.g., be a lyophilized drug. Alternatively, it may be a powder, a tablet, a granulate, etc.

The device comprises locking means adapted to prevent the means for establishing a fluid connection from re-establishing a previously disconnected fluid connection between the reservoir and the vial. This may, e.g., be obtained in a manner which will be described further below. The locking means may, e.g., be adapted to prevent a spike from re-penetrating a septum of a vial and/or a reservoir if the septum/reservoir has previously been penetrated by the spike. Thereby it is prevented that the device is used twice. Furthermore, it may make it possible to additionally use the first forcing means for forcing the reconstituted drug out of the device. The locking means may, e.g., be a releasable snap lock.

The reservoir, the vial, the means for establishing a fluid connection and the forcing means may form an at least substantially integral unit. Thus, in this case these parts together form a single device, e.g. encapsulated by a housing. This has the advantage that the risk of contamination of the various parts of the integral unit, and of the resulting liquid drug, is considerably reduced. Furthermore, the integral unit may be sold as one combined device, thereby ensuring that the liquid in the reservoir matches the dry drug in the vial, in terms of kind of liquid/drug and in terms of amounts. Thus, the device can be manufactured as an ‘all-in-one’ package. Finally, it is possible to design the integral unit in a manner which allows easy operation, e.g. operation using just one hand, and/or operation requiring fewer steps to be performed by the user than corresponding prior art devices. For instance, the steps of positioning containers containing dry drug and liquid can be omitted.

As an alternative, the vial may form a separate and replaceable unit. In this case the device is preferably ‘open’ in the sense that it is possible to gain access to the interior of the device in order to replace a vial.

The device may further comprise releasable locking means adapted to be in a locked and an unlocked position, wherein the releasable locking means, when in its locked position, is adapted to lock the device in a position in which a fluid connection is established between the reservoir and the vial, and in which the first forcing means is forcing liquid from the reservoir to the vial. This can, e.g., be obtained if the reservoir is provided with a movable piston or plunger which may be used for forcing the liquid out of the reservoir and into the vial when the fluid connection has been established. When the piston or plunger has been pushed in, the releasable locking means is moved to the locking position, and thereby the piston or plunger is maintained in the ‘pushed-in’ position without the need for any additional force to be applied by the user. Additionally, the releasable locking means prevents the fluid connection from being interrupted. Thus, the liquid continues to be forced into the vial, without the user having to apply a force, and the user is therefore free to do other things instead, such as directing or guiding a needle into a skin part of the user. Accordingly, this embodiment of the invention is suitable for operation using just one hand.

The releasable locking means may be adapted to be moved from the unlocked position to the locked position by pushing the releasable locking means in a specific direction, and the releasable locking means may be adapted to be moved from the locked to the unlocked position by pushing the releasable locking means in the specific direction, thereby releasing the releasable locking means. According to this embodiment the releasable locking means is operated in a manner similarly to the operation of an ordinary ballpoint pen. Alternatively, the releasable locking means may be provided with a different kind of release mechanism, such as a movable tap or a push button.

The releasable locking means, when in its unlocked position, may be adapted to allow a flow of reconstituted drug to flow from the vial to the reservoir. According to this embodiment, the device is preferably operated in the following manner. Initially, the device is operated to establish a fluid connection between the vial and the reservoir, e.g. including penetrating a septum of the vial, and the first forcing means is operated to start forcing the liquid from the reservoir to the vial via the established fluid connection. The releasable locking means is then moved to the locked position, thereby allowing the liquid to continue to flow into the vial. In case the vial is provided with at least substantially rigid walls, this will cause a pressure to build up in the vial. When all of the liquid has been transferred from the reservoir to the vial, and it has been ensured that the dry drug has been properly reconstituted, the releasable locking means is moved to the unlocked position. Thereby the first forcing means is no longer pushing the liquid towards the vial. Due to the pressure which has built up in the vial, the reconstituted drug will be biased towards the reservoir, and with the force from the first forcing means being absent, the reconstituted drug will flow back into the reservoir, provided that the fluid connection is not interrupted.

The means for establishing a fluid connection may comprise at least a first spike adapted to penetrate the vial, e.g. a septum of the vial. The spike may advantageously be a hollow spike, where the spike penetrates the vial and the hollow part provides access to the interior of the vial once the spike has penetrated the vial.

Alternatively or additionally, the means for establishing a fluid connection may comprise at least a second spike adapted to penetrate the reservoir. In one embodiment the spike may be a double pointed spike, one end of the spike being adapted to penetrate the vial, and the other end being adapted to penetrate the reservoir. Thereby a hollow part of the spike will provide a fluid connection between the reservoir and the vial.

The means for establishing a fluid connection may be arranged inside a closed compartment which is held under sterile conditions. According to this embodiment the fluid connection, e.g. in the form of a double spike, may be held under sterile conditions, even if other parts of the device are not. This is advantageous, because this is the part of the device which gets into contact with the drug. Accordingly, the risk of contamination of the reconstituted drug is even further reduced in this embodiment.

The closed compartment may be delimited at one end by a septum of the vial and at another end by a part of the reservoir. Thereby the parts of the vial and the reservoir which are penetrated when the fluid connection is established are also held under sterile conditions, and the risk of contamination of the reconstituted drug is even further reduced.

The device may further comprise means for delivering a reconstituted drug from the device, e.g. including one or more tubes, a needle, such as a butterfly needle, a syringe, an infusion apparatus, a valve for controlling the flow of reconstituted drug from the device, etc.

The delivering means may comprise second forcing means adapted to force a reconstituted drug towards an outlet opening for delivery of the reconstituted drug. The second forcing means may comprise a piston or a plunger adapted to force the reconstituted drug out of the device. Alternatively, the second forcing means may comprise pumping means and/or any other kind of forcing means which is suitable for forcing the reconstituted drug out of the device. The reconstituted drug may be forced towards the outlet opening directly from the vial. Alternatively, the second forcing means may be adapted to force the reconstituted drug from the reservoir towards the outlet opening.

The second forcing means may be or form part of the first forcing means. In this case the forcing means may advantageously comprise a piston or a plunger positioned in the reservoir. In the embodiment described above, moving the reconstituted drug back into the reservoir causes the piston or plunger to be moved backwards. When the reconstituted drug has been completely transferred to the reservoir, the fluid connection between the reservoir and the vial is preferably interrupted to prevent the reconstituted drug from being transferred back into the vial. If the piston or plunger is subsequently pushed in once again, the reconstituted drug can therefore not move back into the vial. Instead a fluid connection between the reservoir and an outlet opening being connected to delivery means is preferably established, and the reconstituted drug is thereby forced towards the outlet opening, and is thereby delivered from the device.

The delivering means may comprise means for establishing a fluid connection between a part of the device containing reconstituted drug and an outlet opening. The means for establishing a fluid connection to the outlet opening may be adapted to be in a first state in which it is not possible to establish said fluid connection and a second state in which it is possible to establish said fluid connection, and the means for establishing said fluid connection may be adapted to be in the first state during reconstitution of the dry drug and may be adapted to be moved to the second state when the reconstituted drug is ready to be delivered. According to this embodiment it can be ensured that it is not possible to deliver the drug until it is actually ready to be delivered. Thereby it is ensured that drug which has not been properly mixed can not be delivered from the device. This may, e.g., be obtained by arranging a gate valve at the outlet opening. Alternatively or additionally, a movable part may be covering the outlet opening, thereby preventing access through the outlet opening, when the means for establishing a fluid connection to the outlet opening is in the first state. The movable part may then be moved to allow such access when said means is moved to the second state. Alternatively or additionally, the means for establishing a fluid connection to the outlet opening may comprise a threaded portion adapted to receive a luer lock in order to connect tubing, e.g. holding a butterfly needle, to the outlet opening. The threaded portion may, in this case, be locked by a pawl when the means for establishing a fluid connection to the outlet opening is in the first state, thereby preventing the luer lock from being connected to the threaded portion, when the means for establishing a fluid connection to the outlet opening is in the first state. The pawl may be moved to allow a luer lock to be connected to the threaded portion when said means is moved to the second state.

The means for establishing a fluid connection to the outlet opening may be adapted to be automatically moved from the first state to the second state when the reconstituted drug is ready to be delivered. This may, e.g., be obtained by connecting, e.g. a gate valve, a movable part or a pawl, to other parts of the device, e.g. one or more locking means. Thereby it can be ensured that the means for establishing a fluid connection to the outlet opening is operated at an appropriate time during the operation of the device. This embodiment is advantageous, since it prevents or allows a fluid connection to the outlet opening to be established at appropriate times, without the user having to actively do anything. Thereby the risk of introducing human error is minimised.

Alternatively or additionally, the means for establishing a fluid connection to the outlet opening may comprise a third spike adapted to penetrate a septum arranged at or near the outlet opening. The spike is preferably hollow and arranged at or near the outlet opening. Thereby the hollow spike will establish a fluid connection through the septum when penetrating it. The septum may form part of a wall part of the reservoir, in which case a fluid connection is established between the reservoir and the outlet opening. In this case the fluid connection should not be established before the reconstituted drug has been properly retrieved from the vial to the reservoir.

The device may further comprise:
at least one reservoir containing a liquid,
at least two vials, each containing a dry drug, and
means for establishing one or more fluid connections between the reservoir(s) and the vials, thereby causing the dry drug of each of the vials to become reconstituted.

According to this embodiment the dry drug contained in two or more vials may be combined to constitute a dose of reconstituted drug which is larger than a dose corresponding to the dry drug contained in a single vial, i.e. pooling of the contents of the at least two vials is possible. The larger dose may then be delivered from the device. The device may comprise only one reservoir being adapted to supply liquid to each of the vials. In this case liquid may be supplied to each of the vials substantially simultaneously. Alternatively, the device may comprise one reservoir for each vial. In this case the drug in the vials may be reconstituted sequentially.

The device may further comprise means for retrieving reconstituted drug from each of the vials. The reconstituted drug may be retrieved substantially simultaneously from all of the vials, or it may be retrieved sequentially. The reconstituted drug may be retrieved to a common reservoir adapted to contain the full dose, and it may then be delivered to a user from this common reservoir. Alternatively, the reconstituted drug may be delivered directly from the vials to the user, either by retrieving the reconstituted drug substantially simultaneously and leading it directly to an outlet opening, or by sequentially retrieving and delivering drug from the vials. As another alternative, the dry drug in the vials may sequentially be reconstituted, retrieved to a reservoir and delivered to a user from that reservoir.

The device may further comprise means for equalising a pressure build up in the reservoir. Such means may be manually operable, i.e. the user must manually activate the pressure equalising means at an appropriate time, e.g. when reconstituted drug has been retrieved to the reservoir. Alternatively, the pressure equalising means may be automatically operable. According to this embodiment it can be ensured that when the reconstituted drug is to be delivered, it can be delivered in a controlled manner. When reconstituted drug is retrieved from the vial to the reservoir, a high pressure tends to build up in the reservoir. If a fluid connection is subsequently established from the reservoir to an outlet opening in order to allow the reconstituted drug to be delivered, this high pressure will immediately force reconstituted drug out of the device via the established fluid connection and the outlet opening in an uncontrolled manner. This can be avoided by equalising the pressure in the reservoir before the fluid connection is established.

Pressure equalising may, e.g., be obtained by the user pulling a piston backwards. Alternatively, the device may, when the fluid connection to the outlet opening is being established, be positioned in such a manner that the fluid connection is established in an upper region of the reservoir. Thereby air instead of reconstituted drug will be forced out of the device due to the overpressure. Alternatively, a fluid connection may be established between the reservoir and the vial in such a way that air can flow from the reservoir towards the vial, thereby lowering the pressure in the reservoir.

As yet another alternative, the pressure in the reservoir may be equalised by ‘removing material’, thereby increasing the volume of the reservoir. This may, e.g., be obtained by allowing a piston to flex, removing a part of the piston, allowing a septum to flex, etc. This will be described in further detail below with reference to the drawings.

According to a second aspect of the invention the above and other objects are fulfilled by providing a device for establishing a fluid connection between two containers, the device comprising:
a first spike adapted to penetrate a septum of a first container,
a second spike adapted to penetrate a septum of a second container, the first and second spikes being interconnected in such a manner that a fluid connection is established between the first and second containers when the first spike penetrates the septum of the first container and the second spike penetrates the septum of the second container, and
a locking mechanism for locking said spikes in such a manner that re-establishing a previously disconnected fluid connection is prevented.

The device according to the second aspect of the invention may advantageously be arranged in a device according to the first aspect of the invention. The device according to the second aspect of the invention may be or comprise a double needle formed by the first and second spikes. The locking mechanism may be releasable.

According to a third aspect of the invention the above and other objects are fulfilled by providing a method of operating a drug storage and delivery device comprising a reservoir containing a liquid, a vial containing a dry drug and means for establishing a fluid connection between the reservoir and the vial, the method comprising the steps of:
moving the reservoir and the vial towards each other, thereby establishing a fluid connection between the reservoir and the vial,
forcing liquid from the reservoir into the vial, thereby causing the dry drug to become reconstituted,
locking the means for establishing a fluid connection in a position where a fluid connection is established and liquid is being forced from the reservoir to the vial,
unlocking the means for establishing a fluid connection, thereby allowing reconstituted drug to return to the reservoir, and
locking the means for establishing a fluid connection in a position where a fluid connection is disconnected, thereby preventing re-establishing the fluid connection.

The method according to the third aspect of the invention may advantageously be used for operating a device according to the first aspect of the invention, and the remarks set forth above are therefore equally applicable here.

The method may further comprise the steps of:
locking an outlet opening of the drug storage and delivery device, thereby preventing drug from being delivered from the device, and
unlocking the outlet opening after the reconstituted drug has returned to the reservoir, thereby allowing the reconstituted drug to be delivered from the device.

The step of unlocking the outlet opening may be performed automatically. This has been described in detail above.

The method may further comprise the step of automatically performing aspiration via the outlet opening upon unlocking the outlet opening. When the drug is ready to be delivered, a delivery device, such as a needle, e.g. a butterfly needle, possibly connected to the device via suitable tubing, may be inserted at a suitable injection site of a user. According to the present embodiment the device then automatically performs aspiration, i.e. a small amount of body fluid is sucked from the injection site towards the device. This may be used for ensuring that a correct injection site has been chosen (e.g. in a vein or in subcutaneous tissue, etc., depending on the kind of drug). Furthermore, it can be ensured that no air will subsequently be injected.

The method may further comprise the step of equalising a pressure in the reservoir. This has been described in detail above.

According to a fourth aspect of the invention the above and other objects are fulfilled by providing a drug storage and delivery device comprising:
a reservoir containing a liquid,
a vial containing a dry drug,
means for establishing a fluid connection between the reservoir and the vial,
first forcing means for forcing the liquid from the reservoir to the vial, via an established fluid connection, thereby causing the dry drug to become reconstituted,
   wherein the reservoir, the vial, the means for establishing a fluid connection and the forcing means form an at least substantially integral unit.

It should be noted that a person skilled in the art would readily recognise that any feature described in combination with the first aspect of the invention may also be combined with the second, the third and the fourth aspects of the invention, any feature described in combination with the second aspect of the invention may also be combined with the first, the third and the fourth aspects of the invention, any feature described in combination with the third aspect of the invention may also be combined with the first, the second and the fourth aspects of the invention, and any feature described in combination with the fourth aspect of the invention may also be combined with the first, the second and the third aspects of the invention.

### BRIEF DESCRIPTION OF THE DRAWINGS

The invention will now be described in further detail with reference to the accompanying drawings in which

FIG. 1 is a cross sectional view of a drug storage and delivery device according to an embodiment of the invention,

FIG. 2 is a schematic drawing illustrating the operation principle of a releasable locking means of the device of FIG. 1,

FIG. 3 is a schematic drawing illustrating the operation of a locking means of the device of FIG. 1,

FIG. 4 is a perspective view of a double pointed spike for use in a drug storage and delivery device according to an embodiment of the invention,

FIG. 5 is a cross sectional view of a delivery means of a drug storage and delivery device according to an embodiment of the invention,

FIG. 6 is a perspective view of a drug storage and delivery device according to a second embodiment of the invention, the device being operable by one hand,

FIG. 7 is a cross sectional view of the drug storage and delivery device of FIG. 6,

FIG. 8 is a cross sectional view of a drug storage and delivery device according to a third embodiment of the invention,

FIGS. 9-12 illustrate various means for equalising a pressure build up in a reservoir of a drug storage and delivery device according to the invention, and

FIG. 13 is a cross sectional view of a drug storage and delivery device according to a fourth embodiment of the invention.

### DETAILED DESCRIPTION OF THE DRAWINGS

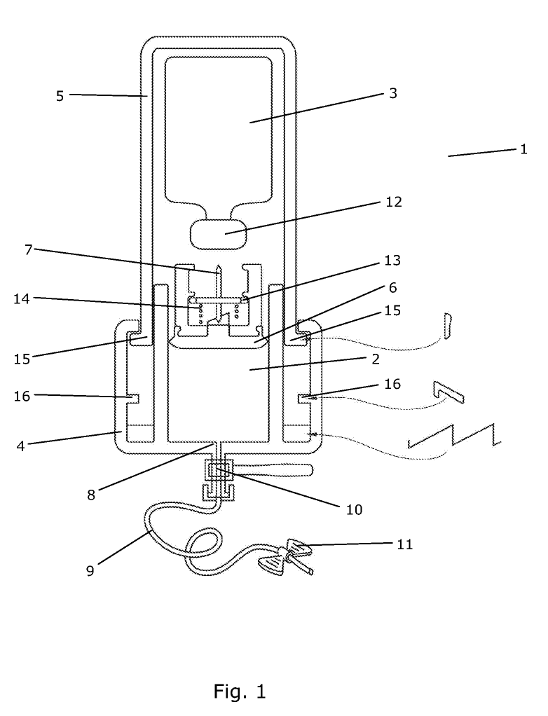

FIG. 1 is a cross sectional view of a device 1 according to an embodiment of the invention. The device 1 comprises a reservoir 2 containing a liquid and a vial 3 containing a dry drug. The reservoir 2 is positioned in a first part 4 of the device 1 and the vial 3 is positioned in a second part 5 of the device 1. The first part 4 and the second part 5 are movable relatively to each other in such a way that the second part 5 may slide inside the first part 4, thereby reducing the height of the device 1.

Between the reservoir 2 and the vial 3 there is positioned a movable plunger 6 having a double pointed hollow needle 7 movably mounted thereon, the hollow needle 7 having its pointed ends directed towards the vial 3 and the movable plunger 6, respectively. The reservoir 2 is further provided with an outlet opening 8 being connected to a tube 9 via a valve 10, the tube 9 being further connected to a butterfly needle 11 for delivering a reconstituted drug from the device 1 to a person.

The device 1 of FIG. 1 is preferably operated in the following manner. Initially the device 1 will be in the position shown in FIG. 1. The reservoir 2 contains a liquid and the vial 3 contains a dry drug. When it is desired to deliver the drug from the device 1, the second part 5 is moved towards the first part 4. Thereby the vial 3 is moved towards the hollow needle 7, and the vial 3 is thereby penetrated by the hollow needle 7. As the second part 5 is moved further towards the first part 4, a neck portion 12 of the vial 3 will press against a disc 13 holding the hollow needle 7. The disc 13 is biased in a direction towards the vial 3 due to spring 14, but it will be forced towards the movable plunger 6 when pressed upon by the neck portion 12. Thereby the hollow needle 7 will penetrate the movable plunger 6, thereby establishing a fluid connection between the reservoir 2 and the vial 3. Furthermore, when the movable plunger 6 has been penetrated by the hollow needle 7, the disc 13 will start pressing against the movable plunger 6. Therefore, by moving the second part 5 even further in the direction of the first part 4 will cause the movable plunger 6 to be moved along. It should be ensured that the valve 10 is in a closed position, thereby preventing the liquid from leaving the reservoir 2 via the outlet opening 8. Accordingly, the movable plunger 6 will cause the liquid to enter the vial 3 via the fluid connection established by the hollow needle 7. When the second part 5 reaches a position where taps 15 on the second part 5 meet corresponding taps 16 on the first part 4, the taps 15, 16 will engage, and the device 1 will be in a locked position where the first 4 and second 5 parts will not be moved relatively to each other. Thus, the device 1 is kept in a position where the fluid connection between the reservoir 2 and the vial 3 is maintained and the plunger 6 forces liquid from the reservoir 2 into the vial 3. The device 1 will stay in this position without any need for the user to apply a force, until the user actively releases the taps 15, 16.

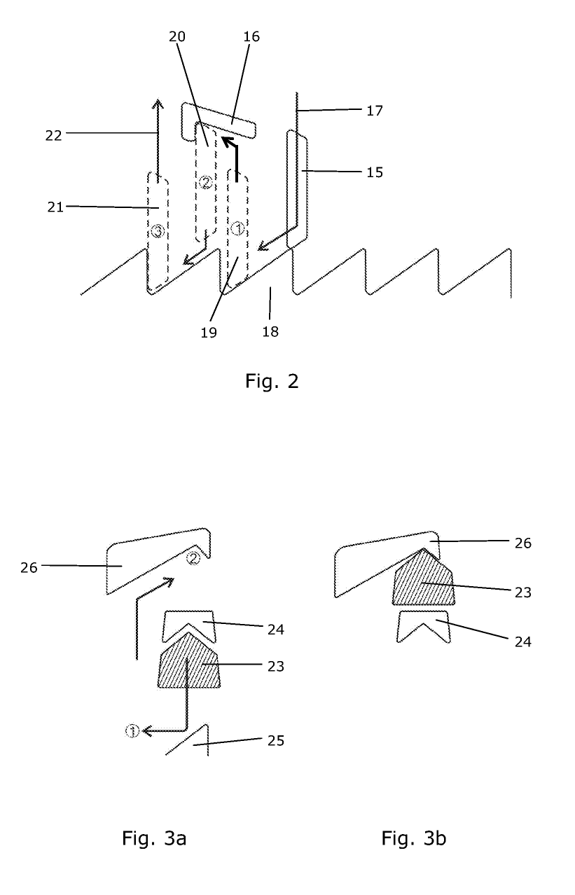

When it has been ensured that the dry drug in the vial 3 has been properly reconstituted, the taps 15, 16 are released. This will be described in further details below with reference to FIG. 2. When the taps 15, 16 have been released, the first 4 and second 5 parts are once again free to move relatively to each other. The liquid which has been transferred to the vial 3 has caused an increase in the pressure inside the vial 3, and the reconstituted drug is therefore inclined to leave the vial 3 via the fluid connection, i.e. it is inclined to enter the reservoir 2. Since the second part 5 is now movable relatively to the first part 4, the movable plunger 6 is also allowed to move. Accordingly, the reconstituted drug will enter the reservoir 2 while moving the movable plunger 6 upwards. When the reconstituted drug has been transferred to the reservoir 2, the fluid connection is interrupted due to movement of the disc 13 caused by the spring 14. The disc 13 will now be in a position where it is not possible for it to move the hollow needle 7 to a position in which the movable plunger 6 is penetrated by the hollow needle 7. This will be described in further detail below with reference to FIG. 3. Accordingly, it is not possible to re-establish the fluid connection between the reservoir 2 and the vial 3.

The valve 10 is now moved to an open position in which liquid is allowed to leave the reservoir 2 via the outlet opening 8. The second part 5, and thereby the vial 3, the disc 13 and the movable plunger 6, is then moved in a direction towards the first part 4. Since there is no fluid connection between the reservoir 2 and the vial 3, and since the valve 10 allows passage of liquid through the outlet opening 8, the movable plunger 6 will force the reconstituted drug through the outlet opening 8, into the tube 9 and further on to the butterfly needle 11 for delivery.

It is possible to operate the device 1 of FIG. 1 using just one hand. This is due to the easy operations as well as the taps 15, 16 for locking the device 1 while liquid is transferred to the vial 3, and the locking mechanism for the disc 13. The operation of the device 1 is also made easier because all the parts necessary for reconstituting and delivering a drug are present in a single unit. Thus, it is not necessary to mount vials or reservoirs containing dry drug and liquid, an operation which normally requires the use of more than one hand. Furthermore, this ensures that the liquid in the reservoir 2 always matches the dry drug in the vial 3, in terms of kind as well as in terms of amount. Thereby the risk of incorrect reconstitution or dosage is minimised.

FIG. 2 is a schematic drawing illustrating the operation principle of a releasable locking means of the device 1 of FIG. 1. FIG. 2 shows a tap 15 positioned on the second part and a tap 16 positioned on the first part. When the second part is moved towards the first part, tap 15 is moved along in a downwards direction as indicated by arrow 17. When the tap 15 reaches tapered portion 18, the second part will be rotated because the tap 15 and the tapered portion 18 abut. The second part will be rotated until position 19 is reached. When pressure on the second part is relieved, tap 15 will move upwards until it engages tap 16 as illustrated by position 20. Thereby the device is locked in the position where liquid is transferred from the reservoir to the vial as described above. After the dry drug in the vial has been properly reconstituted, the second part is once again moved in a direction towards the first part. When tap 15 reaches the tapered portion 18, the second part is once again caused to rotate to position 21. When pressure on the second part is relieved, the tap 15 is free to move upwards without engaging tap 16, as indicated by arrow 22. Accordingly, the device is no longer in the locked position.

FIG. 3 is a schematic drawing illustrating the operation of a locking means of the device 1 of FIG. 1. The locking means is positioned partly on the disc, partly on the movable plunger. Initially, the locking means is in the position illustrated in FIG. 3a, where a first locking part 23, which is positioned on the disc, is engaging a second locking part 24, which is positioned on the movable plunger. When the second part is moved towards the first part as described above, the first locking part 23 is moved downwards along with the disc. When the first locking part 23 reaches tapered portion 25, the disc is caused to rotate because the first locking part 23 and the tapered portion 25 abut. When the pressure on the second part is relieved, the first locking part 23 is moved upwards until it reaches third locking part 26. The disc is once again caused to rotate until the locking means is in the position shown in FIG. 3b. When the second part is once again moved towards the first part, the first locking part 23 will be prevented from moving beyond the position of the second locking part 24 because the first 23 and the second 24 locking part will abut. Thereby the hollow needle is prevented from re-penetrating the reservoir, and the fluid connection between the reservoir and the vial can therefore not be re-established.

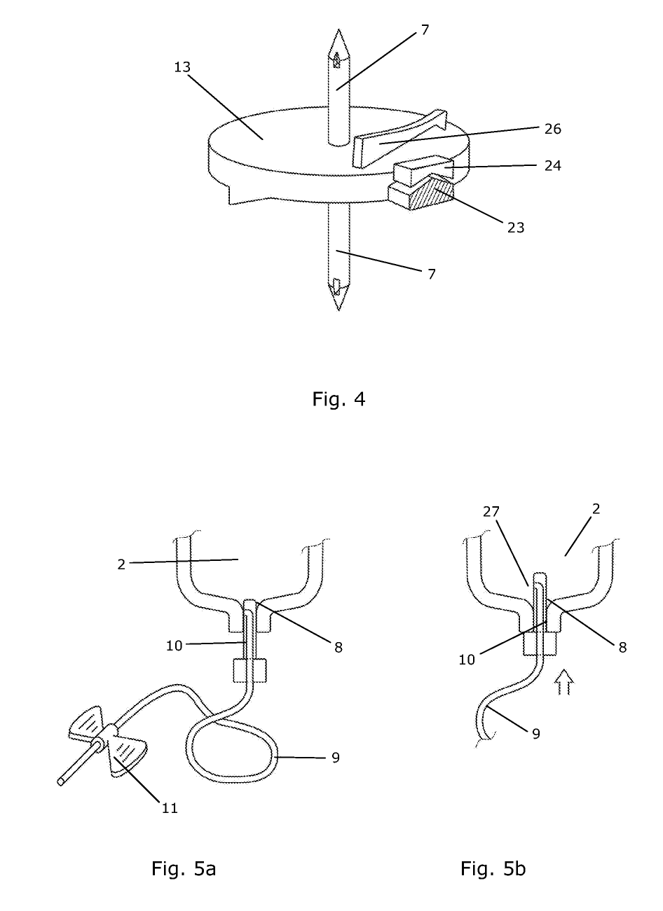

FIG. 4 is a perspective view of a double pointed hollow needle 7 for use in a drug storage and delivery device 1 according to an embodiment of the invention. The hollow needle 7 is mounted on a disc 13 as described above. The relative positions of the first 23, second 24 and third 26 locking parts are shown.

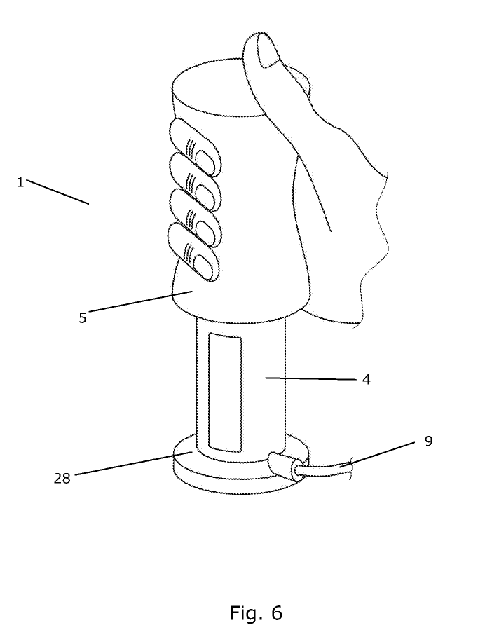

FIG. 5 is a cross sectional view of a delivery means of a drug storage and delivery device 1 according to an embodiment of the invention. FIG. 5 shows just part of the device 1. Thus, the reservoir 2, the outlet opening 8, the valve 10, the tube 9 and the butterfly needle 11 are visible.

In FIG. 5a the valve 10 is in a closed position, i.e. it is not possible for liquid to leave the reservoir 2 via the outlet opening 8. In FIG. 5b the valve 10 is in an open position, i.e. liquid may leave the reservoir 2 via the outlet opening 8. It is possible to move the valve 10 from the closed to the open position using just one hand. When the valve 10 is in the position of FIG. 5a and it is desired to deliver a reconstituted drug, the valve 10 is merely pressed against a relatively flat surface, e.g. a table. Thereby the valve 10 is moved inside the reservoir 2 to the position shown in FIG. 5b. Thereby opening 27 is exposed to the reconstituted drug in the reservoir 2, and a fluid connection is thereby established through which the reconstituted drug may leave the reservoir 2.

FIG. 6 is a perspective view of a drug storage and delivery device 1 according to a second embodiment of the invention. The device 1 of FIG. 6 is operable by one hand. The device 1 comprises a first part 4 and a second part 5 being movable relatively to each other. The operation of the device 1 is similar to the operation of the device 1 of FIG. 1. When the dry drug in the vial (not visible) is to be reconstituted, flat portion 28 is held against a surface, e.g. a table or the thigh of the user. The second part 5 is then held by the user as shown in FIG. 6, and the user presses the second part 5 towards the first part 4, thereby causing liquid to be transferred from the reservoir (not visible) to the vial. In this embodiment, however, the taps for locking the first part 4 relatively to the second part 5 during reconstitution may be omitted because the parts 4, 5 may be held in position by the user pressing the device 1 against the surface. Otherwise the operation is very similar to what is described above, and it will therefore not be described further here.

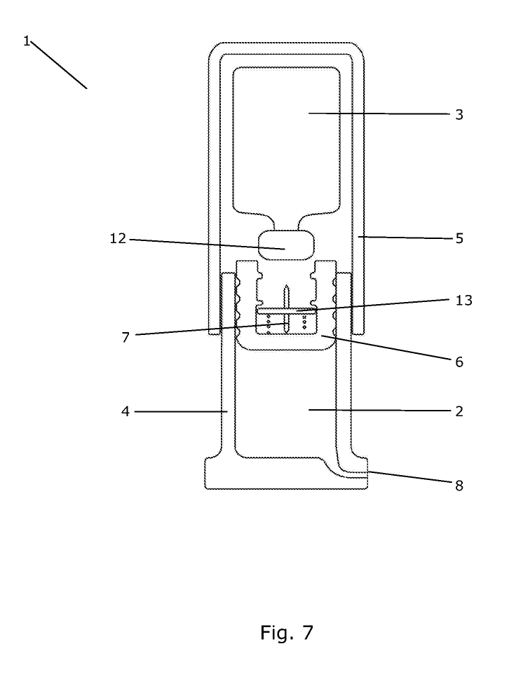

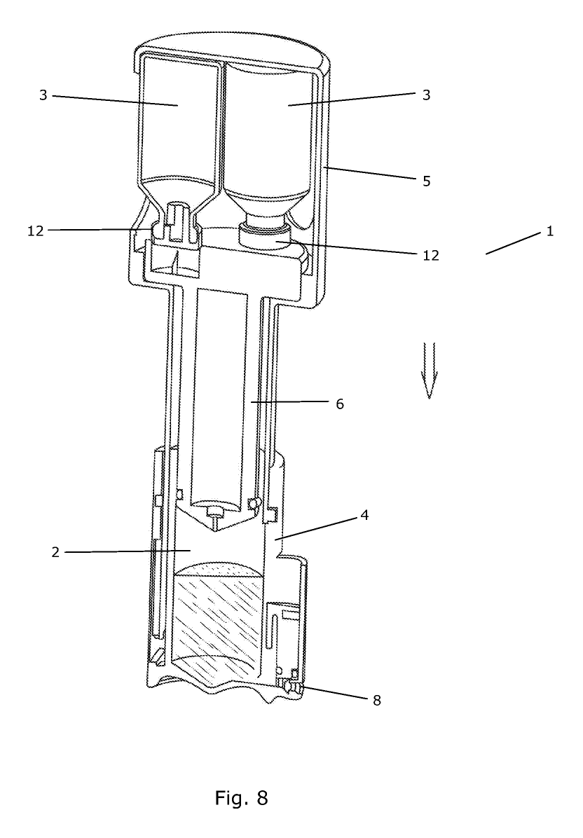

FIG. 7 is a cross sectional view of the drug storage and delivery device 1 of FIG. 6. It is clear from FIG. 7 that the taps have been omitted in this embodiment as described above. Apart from that, the device 1 of FIG. 7 is very similar to the device 1 of FIG. 1.

FIG. 8 is a cross sectional view of a drug storage and delivery device 1 according to a third embodiment of the invention. The device 1 of FIG. 8 comprises three vials 3 arranged in the second part 5 of the device 1. However, only two of the vials 3 are visible in the Figure. The vials 3 are arranged in such a manner that a fluid connection can be established between the reservoir 2 and each of the vials 3. Thereby the dry drug contained in the three vials 3 can be simultaneously reconstituted, and the reconstituted drug may subsequently be retrieved to the reservoir 2 for delivery via the outlet opening 8. Accordingly, a dose of reconstituted drug which exceeds a dose corresponding to the dry drug contained in a single vial 3 can be constituted and delivered by the device 1, i.e. pooling of the contents of the vials 3 can be provided. The device 1 of FIG. 8 is operated essentially as the device 1 of FIG. 1.

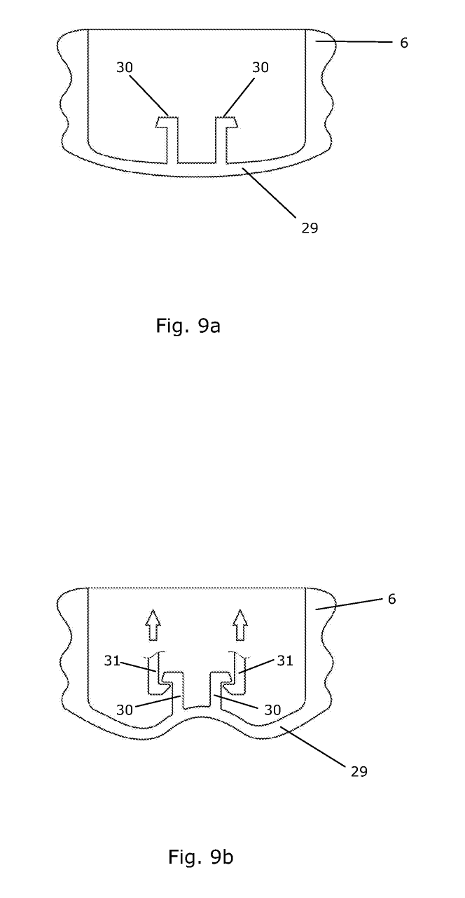

FIG. 9 is a cross sectional view of an end part of a movable piston 6 for use in a device according to the invention. The piston 6 is adapted to be positioned in the reservoir in such a manner that it is capable of forcing liquid from the reservoir to the vial, and preferably also of forcing reconstituted drug from the reservoir towards an outlet opening as described above.

The piston 6 is provided with a flexible end 29 having a pair of engaging parts 30 arranged thereon. In FIG. 9a the piston 6 is shown in a relaxed position, e.g. prior to being penetrated by a hollow needle as described above. After the piston 6 has been penetrated by a needle, the needle is retrieved in order to close the fluid connection between the reservoir and the vial. During this, mating engaging parts 31 formed on the needle or on a holder holding the needle will engage the engaging parts 30. Thereby the flexible end 29 of the piston 6 will be pulled along with the needle, thereby causing the flexible end 29 to bend inwards in a direction away from the reservoir. This situation is illustrated in FIG. 9b. Accordingly the volume of the reservoir is increased, and a pressure build up in the reservoir is thereby equalised.

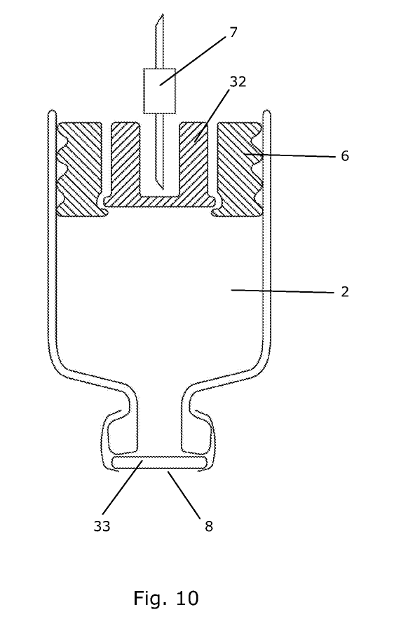

FIG. 10 is a cross sectional view of a reservoir 2 having a movable piston 6 arranged therein. A hollow double needle 7 is arranged in such a manner that it can penetrate the piston 6, thereby establishing a fluid connection between the reservoir 2 and a vial (not shown). Moving the piston 6 will thereby cause liquid from the reservoir 2 to enter the vial as described above. When the reconstituted drug is subsequently retrieved to the reservoir 2 and the needle 7 is pulled back towards the vial, an inner part 32 of the piston 6 is pulled along. Accordingly, the volume of the reservoir 2 is increased, and a pressure build up in the reservoir 2 can thereby be equalised. Subsequently a septum 33 at the outlet opening 8 can be penetrated in order to allow the reconstituted drug to leave the reservoir 2 via the outlet opening 8.

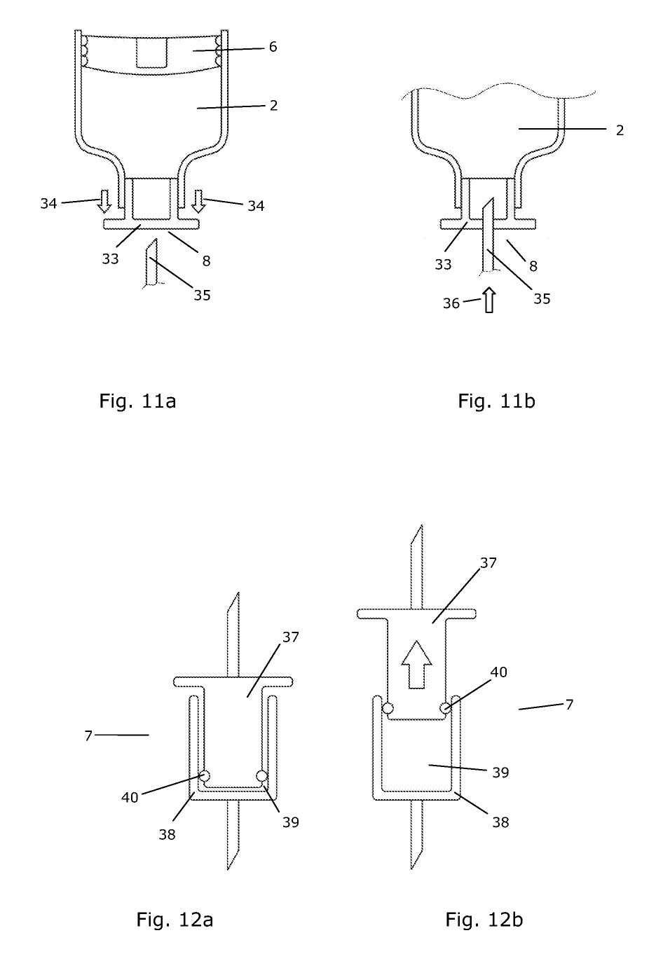

FIG. 11 is a cross sectional view of a reservoir 2 having a movable piston 6 arranged therein. At the outlet opening 8 a septum 33 is arranged in a movable manner. When the reconstituted drug has been retrieved to the reservoir 2 the septum 33 is pulled outwards as indicted by arrows 34 in FIG. 11a. Accordingly, the volume of the reservoir 2 is increased, and a pressure build up in the reservoir 2 can thereby be equalised. Afterwards the septum 33 can be penetrated by a spike 35 being moved in the direction indicated by arrow 36 in FIG. 11b.

FIG. 12 is a cross sectional view of a hollow double needle 7 adapted to establish a fluid connection between a reservoir and a vial as described above. The needle 7 comprises a first spike arranged on a first telescopic part 37 and a second spike arranged on a second telescopic part 38. The first telescopic part 37 is arranged to slide inside the second telescopic part 38. The space 39 between the telescopic parts 37, 38 is sealed by means of gasket 40.

During reconstitution of the dry drug the telescopic parts 37, 38 are positioned as shown in FIG. 12a. When the reconstituted drug has been retrieved to the reservoir as described above, the telescopic parts 37, 38 can be moved away from each other as shown in FIG. 12b. Thereby air from the reservoir is allowed to enter the space 39, and a pressure build up in the reservoir is therefore equalised.

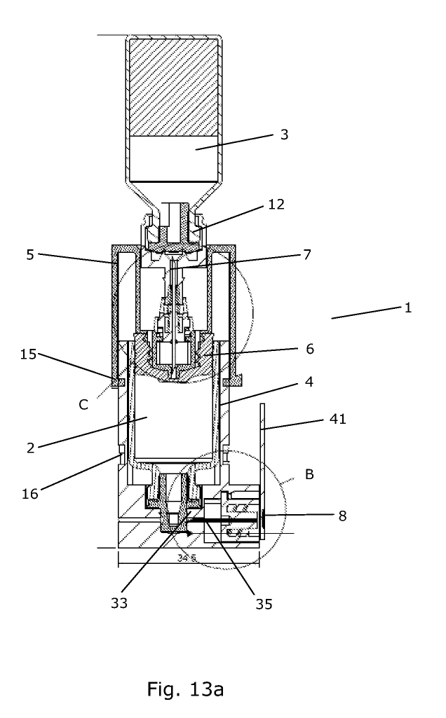

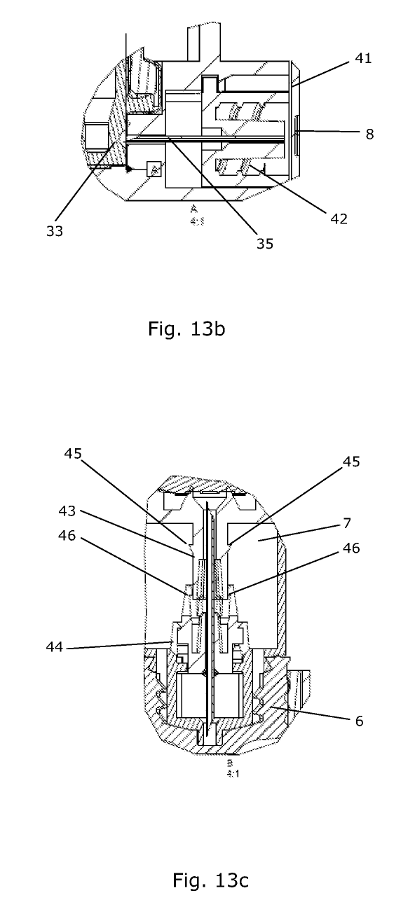

FIG. 13 is a cross sectional view of a drug storage and delivery device 1 according to a fourth embodiment of the invention. The operation of the device 1 is very similar to the operation of the device 1 of FIG. 1, and it will therefore not be described in detail here. FIG. 13a is a full view of the device 1.

The device 1 comprises a hollow double needle 7 adapted to penetrate a septum of a vial 3 and the plunger 6, respectively. FIG. 13c is a detailed view of the hollow double needle 7, and the operation thereof will be described below with reference to FIG. 13c.

The device 1 comprises an outlet opening 8 being provided with a spike 35 adapted to penetrate a septum 33. A locking part 41 is arranged across the outlet opening 8, thereby preventing access through the outlet opening 8. A detailed view of this part of the device 1 is shown in FIG. 13b, and a detailed description is given below.

FIG. 13b is a detailed view of the part of the device 1 indicated by circle B in FIG. 13a. At the outlet opening 8 a threaded portion 42 is provided. The threaded portion is adapted to receive a luer lock arranged on delivery means, e.g. tubing connected to a butterfly needle. In FIG. 13b the locking part 41 is in a locking position, i.e. it is not possible for a luer lock to engage the threaded portion 42. When the reconstituted drug is ready for delivery the locking part 41 can be removed, thereby allowing a luer lock to engage the threaded portion 42. As the luer lock is rotated into position, hollow needle 35 is moved forward, and thereby penetrates septum 33. Thereby a fluid connection is established between the reservoir 2 and the outlet opening 8, and the reconstituted drug can be delivered via this fluid connection.

FIG. 13c is a detailed view of the part of the device 1 indicated by circle C in FIG. 13a. The hollow double needle 7 is arranged on a holder having a first holder part 43 and a second holder part 44. The holder parts 43, 44 are arranged telescopically relatively to each other. When the first part 4 and the second part 5 of the device 1 are moved towards each other and the needle 7 is thereby caused to penetrate the vial 3 and the plunger 6, respectively, the holder parts 43, 44 are also moved relatively to each other in such a manner that the first holder part 43 is moved inside the second holder part 44. Thereby protruding part 45 formed on the first holder part 43 is moved to a position where it engages abutment part 46 formed on the second holder part 44, and the holder parts 43, 44 are thereby prevented from moving relatively to each other when the needle 7 is subsequently retracted from the septum and the plunger 6, thereby disrupting the fluid connection. Accordingly, when the first part 4 and the second part 5 of the device 1 are once again moved towards each other, the needle 7 will not be able to re-establish the disrupted fluid connection, and the plunger 6 can therefore be used for forcing the reconstituted drug out of the device 1 via the outlet opening 8 as described above.

1. A drug storage and delivery device comprising:
a reservoir containing a liquid,
a vial containing a dry drug,
means for establishing a fluid connection between the reservoir and the vial,
first forcing means for forcing the liquid from the reservoir to the vial, via an established fluid connection, thereby causing the dry drug to become reconstituted, and
locking means adapted to prevent the means for establishing a fluid connection from re-establishing a previously disconnected fluid connection between the reservoir and the vial.

2. A device according to claim 1, wherein the reservoir, the vial, the means for establishing a fluid connection and the first forcing means form an at least substantially integral unit.

3. A device according to claim 1, further comprising releasable locking means adapted to be in a locked and an unlocked position, wherein the releasable locking means, when in its locked position, is adapted to lock the device in a position in which a fluid connection is established between the reservoir and the vial, and in which the first forcing means is forcing liquid from the reservoir to the vial.

4. A device according to claim 3, wherein the releasable locking means is adapted to be moved from the unlocked position to the locked position by pushing the releasable locking means in a specific direction, and wherein the releasable locking means is adapted to be moved from the locked to the unlocked position by pushing the releasable locking means in the specific direction, thereby releasing the releasable locking means.

5. A device according to claim 3, wherein the releasable locking means, when in its unlocked position, is adapted to allow a flow of reconstituted drug to flow from the vial to the reservoir.

6. A device according to claim 1, wherein the means for establishing a fluid connection comprises at least a first spike adapted to penetrate the vial.

7. A device according to claim 1, wherein the means for establishing a fluid connection comprises at least a second spike adapted to penetrate the reservoir.

8. A device according to claim 1, wherein the means for establishing a fluid connection is arranged inside a closed compartment which is held under sterile conditions.

9. A device according to claim 8, wherein the closed compartment is delimited at one end by a septum of the vial and at another end by a part of the reservoir.

10. A device according to claim 1, further comprising means for delivering a reconstituted drug from the device.

11. A device according to claim 10, wherein the delivering means comprises second forcing means adapted to force a reconstituted drug towards an outlet opening for delivery of the reconstituted drug.

12. A device according to claim 11, wherein the second forcing means is or forms part of the first forcing means.

13. A device according to claim 10, wherein the delivering means comprises means for establishing a fluid connection between a part of the device containing reconstituted drug and an outlet opening.

14. A device according to claim 13, wherein the means for establishing a fluid connection to the outlet opening is adapted to be in a first state in which it is not possible to establish said fluid connection and a second state in which it is possible to establish said fluid connection, and wherein the means for establishing said fluid connection is adapted to be in the first state during reconstitution of the dry drug and is adapted to be moved to the second state when the reconstituted drug is ready to be delivered.

15. A device according to claim 14, wherein the means for establishing a fluid connection to the outlet opening is adapted to be automatically moved from the first state to the second state when the reconstituted drug is ready to be delivered.

16. A device according to claim 13, wherein the means for establishing a fluid connection to the outlet opening comprises a third spike adapted to penetrate a septum arranged at or near the outlet opening.

17. A device according to claim 1, wherein the first forcing means comprises a movable plunger positioned in the reservoir.

18. A device according to claim 1, the device comprising:
at least one reservoir containing a liquid,
at least two vials, each containing a dry drug, and
means for establishing one or more fluid connections between the reservoir(s) and the vials, thereby causing the dry drug of each of the vials to become reconstituted.

19. A device according to claim 18, further comprising means for retrieving reconstituted drug from each of the vials.

20. A device according to claim 1, further comprising means for equalising a pressure build up in the reservoir.

21. A device for establishing a fluid connection between two containers, the device comprising:
a first spike adapted to penetrate a septum of a first container,
a second spike adapted to penetrate a septum of a second container, the first and second spikes being interconnected in such a manner that a fluid connection is established between the first and second containers when the first spike penetrates the septum of the first container and the second spike penetrates the septum of the second container, and
a locking mechanism for locking said spikes in such a manner that re-establishing a previously disconnected fluid connection is prevented.

22. A device according to claim 21, wherein the locking mechanism is releasable.

23. A method of operating a drug storage and delivery device comprising a reservoir containing a liquid, a vial containing a dry drug and means for establishing a fluid connection between the reservoir and the vial, the method comprising the:
moving the reservoir containing a liquid and the vial containing a dry drug towards each other, thereby establishing a fluid connection between the reservoir and the vial,
forcing liquid from the reservoir into the vial, thereby causing the dry drug to become reconstituted,
locking the means for establishing a fluid connection in a position where a fluid connection is established and liquid is being forced from the reservoir to the vial,
unlocking the means for establishing a fluid connection, thereby allowing reconstituted drug to return to the reservoir, and
locking the means for establishing a fluid connection in a position where a fluid connection is disconnected, thereby preventing re-establishing the fluid connection.

24. A method according to claim 23, further comprising the:
locking an outlet opening of the drug storage and delivery device, thereby preventing drug from being delivered from the device, and
unlocking the outlet opening after the reconstituted drug has returned to the reservoir, thereby allowing the reconstituted drug to be delivered from the device.

25. A method according to claim 24, wherein the step of unlocking the outlet opening is performed automatically.

26. A method according to claim 25, further comprising the step of automatically performing aspiration via the outlet opening upon unlocking the outlet opening.

27. A method according to claim 23, further comprising the step of equalizing a pressure in the reservoir.

28. A drug storage and delivery device comprising:
a reservoir containing a liquid,
a vial containing a dry drug,
means for establishing a fluid connection between the reservoir and the vial,
first forcing means for forcing the liquid from the reservoir to the vial, via an established fluid connection, thereby causing the dry drug to become reconstituted, wherein the reservoir, the vial, the means for establishing a fluid connection and the forcing means form an at least substantially integral unit.
